(19) United States
(12) Patent Application Publication
(10) Pub. No.: US 2005/0192256 A1
(43) Pub. Date: Sep. 1, 2005

(54) Methods of using vitamin D compounds in the treatment of patients undergoing hemodialysis

(76) Inventors: Joel Z. Melnick, Wilmette, IL (US); Laura A. Williams, Gurnee, IL (US); Michael J. Amdahl, Wauconda, IL (US); Jin Tian, Waukegan, IL (US); Steven E. Marx, Hoffman Estates, IL (US); Coleen Hall, Glen Ellyn, IL (US); Dheerendra R. Kommala, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/002,014

(22) Filed: Dec. 2, 2004

Related U.S. Application Data
(60) Provisional application No. 60/530,845, filed on Dec. 18, 2003.
(60) Provisional application No. 60/527,291, filed on Dec. 8, 2003.
(60) Provisional application No. 60/527,391, filed on Dec. 5, 2003.

(51) Int. Cl. A61K 31/59

(52) U.S. Cl. 514/167

(57) ABSTRACT

The subject invention relates to methods of using Vitamin D compounds such as, for example, paricalcitol and calcitriol, in the treatment of patients undergoing hemodialysis. More specifically, administration of Vitamin D compounds appears to reduce the incidence of hospitalization, length of days spent in the hospital and morbidity in treated patients.

The present application claims priority to pending U.S. Provisional Application No. 60/530,845 filed on Dec. 18, 2003 and pending U.S. Provisional Application No. 60/527,291 filed on Dec. 5, 2003. Both priority applications are hereby incorporated in their entirety by reference.

### BACKGROUND OF THE INVENTION

1. Technical Field

The subject invention relates to methods of using Vitamin D compounds such as, for example, paricalcitol and calcitriol, in the treatment of patients undergoing hemodialysis. More specifically, administration of Vitamin D compounds appears to reduce the incidence of hospitalization, length of days spent in the hospital and morbidity in treated patients.

2. Background Information

The prevalence of end-stage renal disease (ESRD) is increasing at an alarming rate. In 2000, end stage kidney disease developed in over 90,000 people in the United States. The current population of patients on dialysis therapy or needing transplantation is 380,000 and projected to be 651,000 patients in 2010. Care for patients with ESRD already consumes more than $18 billion per year in the U.S, a substantial burden for the health care system. New data released in 2003 reported that 19.5 million U.S. adults have chronic kidney disease (CKD), and 13.6 million had Stage 2-5 CKD, as defined by the National Kidney Foundation Kidney Disease Outcomes Quality Initiative (NKF K/DOQI) guideline (“Clinical Practice Guidelines for Bone Metabolism in Chronic Kidney Disease,” American Journal of Kidney Diseases, 42(4), Supp. 3, S1-S201 (October 2003)). Adverse outcomes of chronic kidney disease can often be prevented or delayed through early detection and treatment.

### SUMMARY OF THE INVENTION

The present invention includes a method of reducing the number of hospital admissions in a mammal undergoing hemodialysis (e.g., a human or a domesticated animal) comprising administering to the mammal a Vitamin D compound in an amount sufficient to effect said reduction. The Vitamin D compound may be administered, for example, orally or intravenously. Further, the Vitamin D compound may be, for example, a Vitamin D analog (such as paricalcitol or calcitriol), a Vitamin D metabolite, Vitamin D₂ (or a derivative or analog thereof) or Vitamin D₃ (or a derivative or analog thereof). For example, the Vitamin D compound may be administered in an amount of from about 0.04 mcg/kg to about 0.25 mcg/kg, intravenously, three times/week.

Additionally, the present invention encompasses a method of reducing the length of hospital stays in a mammal undergoing hemodialysis. This method comprises the step of administering a Vitamin D compound to the mammal in an amount sufficient to effect the reduction. Again, the Vitamin D compound may be administered orally or intravenously. The compound may be, for example, a Vitamin D analog (e.g., paracalcitol or calcitriol), a Vitamin D metabolite, Vitamin D₂ (or an analog or derivative thereof) or Vitamin D₃ (or an analog or derivative thereof). Again, the Vitamin D compound may be administered, for example, in an amount of from about 0.04 mcg/kg to about 0.25 mcg/kg, intravenously, three times/week.

### DETAILED DESCRIPTION OF THE INVENTION

Since abnormalities in bone and mineral metabolism are associated with increases in mortality and morbidity (M&M), the K/DOQI work-group developed clinical practice guidelines to manage bone disease in CKD patients. These guidelines were based on studies that examined the relationship between outcomes and serum calcium (serum Ca), phosphate (Phos) and parathyroid hormone (PTH) in prevalent HD patients. The impact of Vitamin D therapy and the K/DOQI guidelines on M&M has not been studied. The evaluation of the interaction between Vitamin D therapy and laboratory values is particularly important.

The subject invention relates to methods of reducing hospital admissions in patients undergoing hemodialysis by administering Vitamin D compounds or Vitamin D analogs to these patients in an amount sufficient to effect such a reduction. More specifically, administration of the compound of interest reduces the incidence of hospitalization stays as well as the length of a hospital stay, associated with the patient's underlying condition, for example, ESRD.

For purposes of the present invention, the term “Vitamin D compound” encompasses compositions which control one or more of the various vitamin D-responsive processes in mammals, e.g., intestinal calcium absorption, bone mobilization, bone mineralization and cell differentiation. Suitable compounds include, but are not limited to, Vitamin D analogs such as, for example, paracalcitol and calcitriol, Vitamin D metabolites, Vitamin D₃ compounds or Vitamin D₃ derivatives such as 1,25-dihydroxyvitamin D₃ (which are created by the use of Vitamin D₃), Vitamin D₂ compounds, Vitamin D₂ derivatives such as 19-nor-1α, 25-dihydroxyvitamin D₂ (made with the use of D₂), cholecalciferol and ergocalciferol and their metabolites, as well as the synthetic cholecalciferol and ergocalciferol analogs which express calcemic or cell differentiation activity (e.g., 5,6 trans-cholecalciferols and 5,6-trans-ergocalciferols, fluorinated cholecalciferols, side chain homologated cholecalciferols, side chain homologated 22 cholecalciferols, side chain-truncated cholecalciferols, the 19-nor cholecalciferols and ergocalciferols and the 10,19-dihydovitamin D compounds), 1-hydroxyvitamin D₃, 1-hydroxyvitamin D₂, 1,25-dihydroxyvitamin D₃, 1,25-dihydroxyvitamin D₂, 25-hydroxyvitamin D₃, 25-hydroxyvitamin D₂, 24,24-difluoro-25-hydroxyvitamin D₃, 24,24-difluoro-25-dihydroxyvitamin D₃, 2-fluoro-25-hydroxyvitamin D₃, 2-fluoro-1,25-dihydroxyvitamin D₃, 26, 26, 26, 27, 27, 27-hexafluoro-25-hydroxyvitamin D₃, 26, 26, 26, 27, 27, 27-hexafluoro-1,25-hydroxyvitamin D₃, 24-25-dihydroxyvitamin D₃, 14,25-trihydroxyvitamin D₃, 5,26-dihydroxyvitamin D₃, 15,26-trihydroxyvitamin D₃, 3,25-dihydroxyvitmain D₃, 23,25,26-trihydroxyvitamin D₃ and the corresponding 1-hydroxylated forms, 25-hydroxyvitamin D₃, -26,23-lacton and its 1-hydroxylated derivative, the side chain, nor, dinor, trinor and tetranor-analogs of 25-hydroxyvitamin D₃, and of, -dihydroxyvitamin D₃, 1-hydroxypregnacalciferol, and its homo and dihomo derivatives, 1,25-dihydroxy-24-20i-vitamin D₂, 24-homo-1,25,dihydroxyvitmain D₃, 24-dihomo-1,25-dihydroxyvitmain D₃, 24-trihomo-1,25-dihydroxyvitamin D₃, and the corresponding 26- or 26,27-homo, dihomo or trihomo analogs of 1,25-dihydroxyvitmain D₃, as well as the corresponding 19-nor compounds of those listed above.

The compound can be administered in a variety of ways. For example, as noted above, the compound can be administered orally or intravenously. Alternatively, the compound can be released from a surgical or medical device or implant, such as a stent (i.e., a drug coated stent), a graft (i.e., such as a vascular access or bypass graft), catheters, sutures, prosthesis and the like. The device or implant can be coated, embedded or impregnated with the compound. The compound may also be formed as a film.

The compound to be administered pursuant to the method described herein can be formulated following techniques known in the art and suitable for administration via the selected route. For example, any pharmaceutically acceptable formulation containing the compound may be used, including, but not limited tablets, solutions, powders, suspension, creams, aerosols, etc. Any pharmaceutically acceptable carriers known or anticipated in the art may be added to the formulation.

The Vitamin D compound can be formulated following techniques known in the art and suitable for administration via the selected route. For example, oral capsules are disclosed in U.S. Pat. No. 4,341,774 and formulations suitable for intravenous administration are disclosed in U.S. Pat. No. 4,308,264 and WO 96/36340. Preferably, the Vitamin D compound is administered in a therapeutically effective amount of from about 0.04 to about 0.24 mcg/kg, intravenously, 3 times per week, depending upon the vitamin D compound administered.

The Vitamin D compound (preferably paricalcitol (also known as 19-nor-1α,3β,25-trihydroxy-9,10-secoergosta-5(Z); 7(E),22(E)-triene, 1α,25-dihydroxy-19-norergocalciferol, 19-nor-1α,25-dihydroxyvitamin D₂ and 1α,25-dihydroxyl-19-nor-vitamin D₂) or calcitriol (also known as 1α,25-dihydroxy-vitamin D₃) can be formulated following techniques known in the art. Preferably, the Vitamin D compound is administered in a therapeutically effective amount of from about 0.04 to about 0.24 mcg/kg, intravenously, 3 times per week, depending upon the Vitamin D compound administered.

The present invention may be illustrated by the use of the following non-limiting examples:

### EXAMPLE I

### Examination of the Relationship Between Vitamin D Therapy And K/DOQI Guidelines on Hospitalization Outcomes

An examination was made of the relationship between Vitamin D therapy and K/DOQI guidelines on hospitalization outcomes, as a surrogate for morbidity, in hemodialysis (HD) patients. The cohort consisted of U.S. patients who were new to HD during the period from January 1999 to December 2001 and who received paricalcitol (P), calcitriol (C) or no vitamin D (NoD) therapy for at least 180 days. To allow for patients to be stabilized after initiation to Vitamin D or NoD therapy, baseline laboratory values were defined over the initial 90-180 days of the observation period; mean values over this baseline period were assessed relative to K/DOQI guidelines. Patients with baseline laboratory measurements within K/DOQI guidelines served as reference groups. An interaction term was created to examine the relationship between Vitamin D therapy and the K/DOQI baseline assessment. Outcomes were defined by annual hospitalizations (HOSP) and hospital days (DAYS). Analysis was performed using negative binomial regression modeling.

Regression analyses revealed that the NoD group (N=3007) experienced significantly more HOSP and DAYS compared with either P (N=1318) or C (N=1432) groups, p<0.05 for both. No significant relationship was found between baseline laboratory values, D therapy and HOSP. However, for DAYS, the interaction terms to assess the NoD group relative to baseline K/DOQI guidelines indicated fewer DAYS for patients outside guidelines for PTH, Ca x P product and Ca; NoD patients outside of guideline for Phos experienced greater DAYS (see Table 1 below). The K/DOQI guideline interaction assessment revealed only a significant increase (40%) in DAYS for the C patients outside of K/DOQI guidelines for Phos, p<0.05. Interestingly, for the P group, there was no significant difference in outcomes for patients within K/DOQI guidelines compared to those outside of guidelines.

TABLE 1
Annual Hospitalization Differences
for NoD Patients Outside K/DOQI

| | % outside | % difference in | P |
|---|---|---|---|
| Lab Variable | K/DOQI | hospitalizations | Value |
| IPTH | 82 | −13 | <0.0005 |
| Ca | 8 | −20 | <0.05 |
| Phos | 51 | 17 | <0.01 |
| Ca × P | 32 | −15 | <0.05 |

In conclusion, K/DOQI target laboratory values examined at the start of chronic HD predict morbidity only for patients not receiving IV Vitamin D therapy. For those patients on IV Vitamin D therapy, K/DOQI laboratory values predict morbidity in only those patients receiving C who are outside Phos target. For patients receiving P, who experience fewest hospitalization outcomes, K/DOQI guidelines do not associate with differences in outcomes. Thus, since Vitamin D therapy associates with decreased M&M, new K/DOQI bone and mineral guidelines should be considered for patients receiving Vitamin D.

### EXAMPLE II

### Association of Citamin D Therapy, Serum PTH and CaxP Product with Morbidity Risk in Hemodialysis Patients

Elevated PTH and CaxP levels have been considered risk factors for increased mortality and morbidity (M&M) in HD patients. However, the observational studies that have predicted such risks did not evaluate Vitamin D therapy as a possible confounder. Recent data have shown that a Vitamin D analog, paricalcitol (Zemplar®), was associated with survival benefit in HD patients, and similar benefits have been shown with paricalcitol as it relates to morbidity (hospitalizations). To date, the relationship between M&M, PTH, CaxP product and the impact of Vitamin D therapy, however, has not been addressed. Thus, by quintiles, an analysis was made of the baseline distribution of serum iPTH, and CaxP product levels in HD patients who received paricalcitol (P), calcitriol (C) or no Vitamin D (NoD) therapy in regard to their relationship with hospitalization outcomes as defined by hospitalizations per year (HOSP) and hospital days per year (DAYS).

The cohort consisted of 11,340 adult patients new to HD between January 1999 and November 2001; there were 2,316 patients treated with P; 2,299 patients treated with C and 6,725 patients NoD. Negative binomial regression analysis revealed that the P group was associated with 90% and 72% fewer DAYS compared to NoD and C groups (p<0.0001 and p=0.0036, respectively).

An investigation of the interaction effects between laboratory quintiles and Vitamin D therapy was completed. There were no significant associations between quintiles of PTH, CaxP and hospital outcomes in patients who received or C except for significantly fewer DAYS in the 2^{nd} quintile of CaxP for C compared to the reference group (3^{rd} quintile). In the NoD group, those in the lowest quintile for CaxP (<31 mg/dL²) or the two highest quintiles for CaxP (44.7-54.3 and <54.3 mg/dL²) or highest quintile for PTH (>473 pg/mL) were associated with significantly more HOSP compared to the reference group of each laboratory parameter (p<0.0001, p=0.0009, p=0.0231 and p=0.0312, respectively). In addition, NoD patients in the highest quintiles for PTH (<473 pg/mL) or the lowest quintile for CaxP (<31 mg/dL²) were associated with more DAYS than the reference groups (p=0.0012 and p=0.0015, respectively).

In conclusion, the above study supports the association of serum PTH and CaxP product with morbidity for patients not receiving vitamin D. For patients receiving calcitriol, low CaxP levels were significantly associated with morbidity. For patients receiving paricalcitol, PTH or CaxP were not associated with morbidity. This analysis suggests that Vitamin D therapy, more specifically paricalcitol, is a confounder for the association between morbidity and PTH or CaxP product, and that the benefits or paricalcitol extend beyond PTH or mineral control.

1. A method of reducing the number of hospital admissions in a mammal undergoing hemodialysis comprising administering to said mammal a Vitamin D compound in an amount sufficient to effect said reduction.

2. The method of claim 1 wherein said Vitamin D compound is administered orally or intravenously.

3. The method of claim 2 wherein said Vitamin D compound is selected from the group consisting of calcitriol, a Vitamin D analog, a Vitamin D metabolite, Vitamin D₂ and Vitamin D₃.

4. The method of claim 3 wherein said Vitamin D analog is paricalcitol.

5. The method of claim 3 wherein said Vitamin D compound is administered in an amount of from about 0.04 mcg/kg to about 0.25 mcg/kg, intravenously, three times/week.

6. A method of reducing the length of hospital stays in a mammal undergoing hemodialysis comprising administering to said mammal a Vitamin D compound in an amount sufficient to effect said reduction.

7. The method of claim 5 wherein said Vitamin D compound is administered orally or intravenously.

8. The method of claim 6 wherein said Vitamin D compound is selected from the group consisting of calcitriol, a Vitamin D analog, a Vitamin D metabolite, Vitamin D₂ and Vitamin D₃.

9. The method of claim 8 wherein said Vitamin D analog is paricalcitol.

10. The method of claim 8 wherein said Vitamin D compound is administered in an amount of from about 0.04 mcg/kg to about 0.25 mcg/kg, intravenously, three times/week.
